(19) United States
(12) Patent Application Publication
(10) Pub. No.: US 2015/0048830 A1
(43) Pub. Date: Feb. 19, 2015

(54) Magnetic Resonance Imaging Apparatus and Bed Board Assembly

(71) Applicants: Jian Hua Pei, Shenzhen (CN); Wen Qiang You, Shenzhen (CN)

(72) Inventors: Jian Hua Pei, Shenzhen (CN); Wen Qiang You, Shenzhen (CN)

(21) Appl. No.: 14/444,761

(22) Filed: Jul. 28, 2014

(30) Foreign Application Priority Data
Aug. 14, 2013 (CN) CN 201310354115.6

(51) Int. Cl. A61B 5/055

(52) U.S. Cl. CPC A61B 5/0555
USPC 324/321

(57) ABSTRACT

An MRI apparatus and a bed board assembly with illumination are described. The MRI apparatus includes: a frame with a cavity; a support rail disposed in the cavity; a bed board, which enters or exits the cavity moveably via the support rail; and an illumination unit disposed in a front portion of the bed board, for emitting light to provide illumination when the MRI apparatus is performing a scan. Space inside the cavity may be saved, and an examination subject may have the impression of being in a more open and brighter space, alleviating the subject's discomfort or fear effectively.

This application claims the benefit of CN 2013 10354115.6, filed on Aug. 14, 2013, which is hereby incorporated by reference in its entirety.

### TECHNICAL FIELD

The present embodiments relate to magnetic resonance imaging (MRI), in particular to an MRI apparatus and a bed board assembly.

### BACKGROUND

Magnetic resonance imaging (MRI) is an imaging technology involving biomagnetism and nuclear spin. MRI has grown rapidly with the development of computer technology, electronic circuit technology and superconductor technology. MRI uses a magnetic field and radio frequency (RF) pulses to cause nutation of processing hydrogen nuclei (i.e. H+) in human tissue to generate RF signals, which are processed by computer to form an image. When an object is placed in the magnetic field, if the object is irradiated with appropriate electromagnetic waves to make nuclei resonate, and the electromagnetic waves released thereby are then analyzed, it is possible to discover the positions and types of the atomic nuclei that form the object, and on this basis a precise three-dimensional image of the interior of the object may be drawn. For example, MRI may be used to scan a human brain to obtain a moving image of contiguous slices, beginning at the top of the head and going all the way to the base thereof.

In existing MRI apparatuses, the cavity in which the examination subject is received is cramped and quite dim. During an MRI examination of long duration, such an environment is likely to inflict psychological stress on the examination subject, with claustrophobic people, children and other special groups being especially likely to suffer a major psychological impact.

FIG. 1 is a schematic diagram of part of an MRI apparatus. The imaging apparatus includes a frame with a cylindrical cavity, a support rail disposed inside the cavity, and a bed board that enters or exits the cavity moveably via the support rail. An illuminating light is installed on the frame housing at the head or tail end.

However, the cavity in which the examination subject is examined is quite deep, and since only the head or tail area of the frame has an illuminating light installed, the light level deep within the cavity remains quite low, while the intensity of illumination is not uniform.

### SUMMARY AND DESCRIPTION

The scope of the present invention is defined solely by the appended claims and is not affected to any degree by the statements within this summary.

The embodiments present an MRI apparatus that may illuminate a cavity well.

The embodiments present a bed board assembly that may illuminate a cavity well.

The technical solution of the embodiments is as follows:

An MRI apparatus, including: a frame with a cavity; a support rail disposed in the cavity; a bed board, which enters or exits the cavity moveably via the support rail; and an illumination unit disposed in a front portion of the bed board.

The illumination unit is disposed on left and right sides of the front portion of the bed board, and emits light towards a cavity wall of the cavity.

The left and right sides of the front portion of the bed board are each provided with a first slot, in which the illumination unit is embedded.

The direction in which light is emitted by the illumination unit makes an included angle with the horizontal direction, the included angle being within the range of 15 degrees to 60 degrees.

Reflective film is stuck to the cavity wall of the cavity.

The illumination unit is disposed on a top surface of the front portion of the bed board, and emits light towards a cavity wall of the cavity.

The top surface of the front portion of the bed board is provided with a second slot in which the illumination unit is embedded.

The illumination unit is disposed at a front bed head in the front portion of the bed board, and emits light towards a cavity wall of the cavity.

Further included are: a second illumination unit disposed on a front/rear housing of the frame; and/or a third illumination unit disposed on the support rail; and/or a fourth illumination unit disposed on the cavity. The illumination unit is an LED light source.

A bed board assembly, including: a bed board; a support frame for supporting the bed board and matching a support rail in a cavity, the bed board entering or exiting the cavity moveably via the support rail; and an illumination unit disposed in a front portion of the bed board. The illumination unit is disposed on the left and right sides of the front portion of the bed board, a top surface of the front portion of the bed board, or a front bed head in a front region of the bed board.

It can be seen from the above technical solution that in some embodiments, an MRI apparatus is presented, including: a frame with a cavity; a support rail disposed in the cavity; a bed board, which enters or exits the cavity moveably via the support rail; and an illumination unit disposed in a front region of the bed board, for emitting light to provide illumination when the MRI apparatus is performing a scan. As can be seen, when the embodiments are applied, having the illumination unit disposed in a front region of the bed board enables space to be saved inside the cavity, and enables the examination subject to have the impression of being in a more open and brighter space; furthermore, light beams provide dynamic and sustained illumination as the examination subject's body moves deep into the cavity interior, and so may alleviate the subject's discomfort or fear effectively.

### DESCRIPTION OF THE ACCOMPANYING DRAWINGS

FIG. 1 is a schematic diagram of part of an MRI apparatus in the prior art.

FIG. 2 is a schematic diagram of the structure of the bed board according to one embodiment.

FIG. 3 is a schematic diagram of part of an MRI apparatus in a first embodiment.

FIG. 4 is a magnified schematic diagram of part of an MRI apparatus in the first embodiment.

FIG. 5 is a schematic diagram showing light emission in part of an MRI apparatus in the first embodiment.

FIG. 6 is a schematic diagram of part of an MRI apparatus in a second embodiment.

FIG. 7 is a perspective schematic diagram showing light emission in an MRI apparatus in the second embodiment.

FIG. 8 is a schematic diagram showing light emission in an MRI apparatus in the second embodiment.

### DETAILED DESCRIPTION OF THE PREFERRED EMBODIMENTS

The present embodiments are explained in further detail below with reference to the accompanying drawings, in order to clarify the technical solution and advantages thereof. It should be understood that the particular embodiments described here are intended merely to illustrate the present invention elaboratively, not to define the scope of protection thereof.

First, the structure of the bed board shall be explained by way of demonstration.

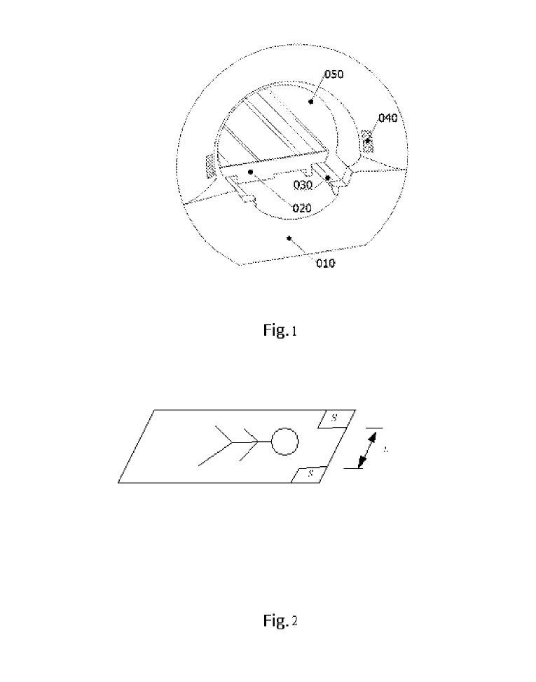

FIG. 2 is a schematic diagram of the structure of the bed board of one embodiment.

As FIG. 2 shows, a person lies on the bed board, with the region of the bed board close to the person's head being the front of the bed board. The front of the bed board may specifically include: left and right side regions located on either side of the head (i.e. the regions marked with the letter S in the figure) and a front bed head region (i.e. the length marked with the letter L in the figure). The dimensions of the left and right side regions and front bed head region shown in the figure are no more than exemplary, and are not intended to define the embodiments of the present invention.

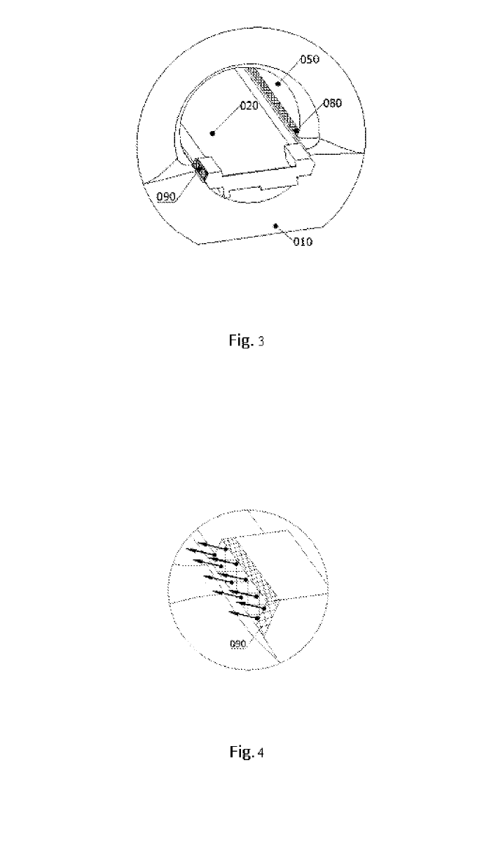

FIG. 3 is a schematic diagram of part of an MRI apparatus in a first embodiment.

As FIG. 3 shows, the MRI apparatus includes: a frame with a cavity (050); a support rail disposed in the cavity (050); a bed board (020) which enters or exits the cavity (050) moveably via the support rail; and an illumination unit (090) disposed in a front region of the bed board (020), for emitting light to provide illumination when the MRI apparatus is performing a scan.

The support rail is generally two parallel rails disposed on an inside wall of the cavity (050). The length direction of the rails is parallel to a center line of the cavity (050). The support rail may also be any component capable of guiding and supporting the bed board (020).

The bed board (020) enters or exits the cavity (050) moveably via the support rail, for the purpose of moving an examination subject into or out of the cavity (050). In general, examination subjects always enter the cavity (050) head first, therefore the front region of the bed board (020) is the region of the bed board that is moved into the cavity (050) first when the examination subject enters the cavity (050); and the region of the bed board that leaves the cavity (050) last when the examination subject leaves the cavity (050).

Preferably, the front region of the bed board (020) takes up around 15% of the total length of the bed board. The front region of the bed board (020) may encompass the head of the examination subject, so that the illumination provided in the front region of the bed board (020) may give the examination subject a better experience.

In FIG. 3, the illumination unit (090) is disposed on side faces on the edges of the bed on the left and right sides of the front region of the bed board (020), and emits light towards the left and right cavity walls of the cavity (050), respectively. FIG. 3 shows an illumination unit (090) located on a side face on the left edge of the bed, but in fact, an illumination unit (090) may also be included on the side face on the right edge of the bed.

When the bed board (020) enters or exits the cavity (050) moveably via the support rail, the illumination unit (090) may also enter or exit the cavity (050) moveably along with the bed board (020). Therefore, light beams provide dynamic and sustained illumination as the examination subject's body moves deep into the cavity interior, and so can alleviate the subject's discomfort or fear effectively.

Preferably, slots may be provided in the side faces on the left and right edges of the bed, with the illumination units (090) being embedded in the slots. Embedding enables space to be saved inside the cavity and enables the examination subject to have the impression of being in a more open and brighter space, alleviating the subject's discomfort or fear effectively.

Preferably, the direction in which light is emitted by the illumination unit (090) makes a certain included angle with the wall of the cavity (050), thereby enabling light beams emitted by the illumination unit (090) to be fully and uniformly reflected from the wall of the cavity (050), with a reduction in the intensity of the reflected light beams.

More preferably, the included angle may be within the range of 15 degrees to 60 degrees, for example, 15 degrees to 30 degrees.

Reflective film (080) may also be stuck to the left and right cavity walls of the cavity (050), for the purpose of reflecting light from the illumination unit (090).

The illumination unit (090) may be any light source, for example, a light-emitting diode (LED) light source. Moreover, the illumination unit (090) may also include a light source controller, for regulating the intensity of the light beams emitted by the illumination unit (090).

The electricity needed by the illumination unit (090) may be supplied by a circuit in the MRI apparatus, by an external power supply, or by an internal battery.

FIG. 4 is a magnified schematic diagram of part of an MRI apparatus in the first embodiment. As FIG. 4 shows, the direction in which light is emitted from the illumination unit (090) makes a certain included angle with a wall of the cavity (050).

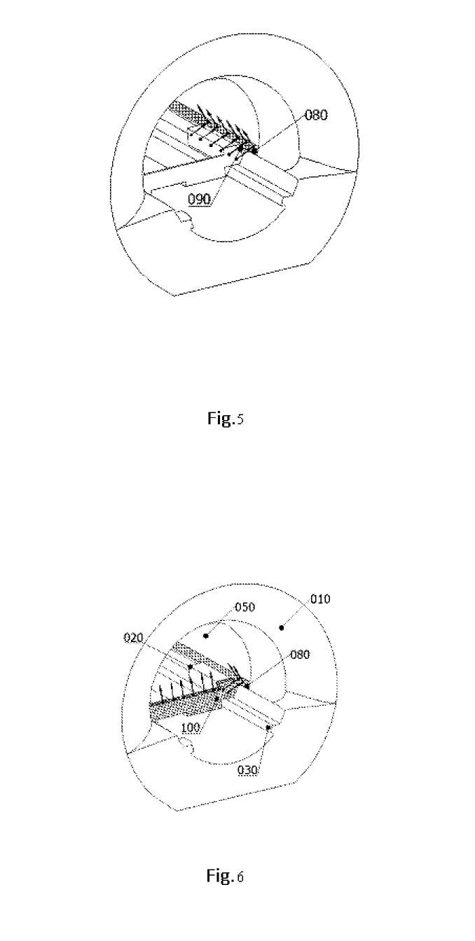

FIG. 5 is a schematic diagram showing light emission in part of an MRI apparatus in the first embodiment. As FIG. 5 shows, reflective film (080) is stuck to the left and right cavity walls of the cavity (050), for the purpose of reflecting light from the illumination unit (090).

In this embodiment, a second illumination unit may be further provided on a front/rear housing (010) of the frame; a third illumination unit may be further provided on the support rail; and/or a fourth illumination unit may be further provided on the cavity (050).

When the bed board is withdrawn from the MRI apparatus, the various illumination units in the cavity are switched off. When the bed board is moved into the MRI apparatus, the various illumination units in the cavity are switched on.

In another embodiment, the left and right edges of the bed may also each be provided with a slot in the vertical direction, with illumination units (090) embedded in the slots. Thus, the illumination units (090) may emit light beams directly onto an upper wall of the cavity (050). The light beams are scattered uniformly by the hemispherical upper wall of the cavity (050).

In another embodiment, the illumination unit (090) may also be disposed at a front bed head in a front region of the bed board, with emitted light directions each facing the upper cavity wall of the cavity. Similarly, light beams may be emitted directly onto the upper wall of the cavity (050). The light beams are scattered uniformly by the hemispherical upper wall of the cavity (050).

FIG. 6 is a schematic diagram of part of an MRI apparatus in a second embodiment.

As FIG. 6 shows, the MRI apparatus includes: a frame with a cavity (050); a support rail (030) disposed in the cavity (050); a bed board (020) which enters or exits the cavity (050) moveably via the support rail; and an illumination unit (100) disposed at a front bed head in a front region of the bed board (020), for emitting light to provide illumination when the MRI apparatus is performing a scan.

Since the front bed head in a front region of the bed board (020) is generally longer than the left and right edges of the bed, a better illumination effect may be achieved by disposing the illumination unit (100) at the front bed head in the front region of the bed board (020).

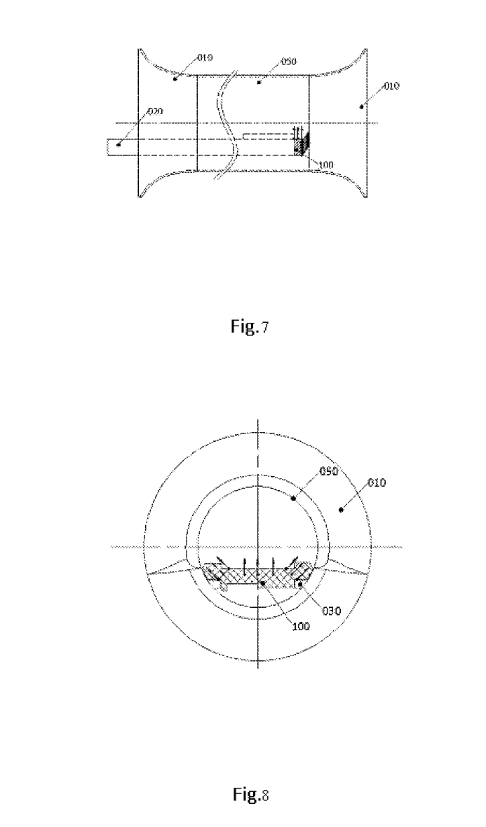

FIG. 7 is a perspective schematic diagram showing light emission in an MRI apparatus in the second embodiment. FIG. 8 is a schematic diagram showing light emission in an MRI apparatus in the second embodiment.

Similarly, in this embodiment, a second illumination unit may be further provided on a front/rear housing (010) of the frame; a third illumination unit may be further provided on the support rail (030); and/or a fourth illumination unit may be further provided on the cavity (050).

When the bed board is withdrawn from the MRI apparatus, the various illumination units in the cavity are switched off. When the bed board is moved into the MRI apparatus, the various illumination units in the cavity are switched on.

Based on the above analysis, the embodiments also present a bed board assembly. The bed board assembly includes: a bed board; a support frame for supporting the bed board and matching a support rail in a cavity, the bed board entering or exiting the cavity moveably via the support rail; and an illumination unit disposed in a front portion of the bed board.

Preferably, the illumination unit may be disposed on the left and right sides of the front portion of the bed board, the top surface of the front portion of the bed board, or a front bed head in a front region of the bed board.

In summary, the MRI apparatus of one of the present embodiments includes: a frame with a cavity; a support rail disposed in the cavity; a bed board, which enters or exits the cavity moveably via the support rail; and an illumination unit disposed in a front region of the bed board, for emitting light to provide illumination when the MRI apparatus is performing a scan. As can be seen, when one of the embodiments are applied, having the illumination unit disposed in a front region of the bed board enables space to be saved inside the cavity and enables the examination subject to have the impression of being in a more open and brighter space. Furthermore, light beams provide dynamic and sustained illumination as the examination subject's body moves deep into the cavity interior, and so can alleviate the subject's discomfort or fear effectively.

The above embodiments are merely preferred embodiments of the present invention, and are not intended to define the scope of protection thereof. Any amendments, equivalent substitutions or improvements etc. made within the spirit and principles of the present invention should be included in the scope of protection thereof.

It is to be understood that the elements and features recited in the appended claims may be combined in different ways to produce new claims that likewise fall within the scope of the present invention. Thus, whereas the dependent claims appended below depend from only a single independent or dependent claim, it is to be understood that these dependent claims can, alternatively, be made to depend in the alternative from any preceding or following claim, whether independent or dependent, and that such new combinations are to be understood as forming a part of the present specification.

While the present invention has been described above by reference to various embodiments, it should be understood that many changes and modifications can be made to the described embodiments. It is therefore intended that the foregoing description be regarded as illustrative rather than limiting, and that it be understood that all equivalents and/or combinations of embodiments are intended to be included in this description.

1. An MRI apparatus comprising:
a frame with a cavity;
a support rail disposed in the cavity;
a bed board that enters or exits the cavity moveably via the support rail; and
an illumination unit disposed in a front portion of the bed board.

2. The MRI apparatus as claimed in claim 1, wherein the illumination unit is disposed on left and right sides of the front portion of the bed board, and emits light towards a cavity wall of the cavity.

3. The MRI apparatus as claimed in claim 2, wherein the left and right sides of the front portion of the bed board are each provided with a first slot in which the illumination unit is embedded.

4. The MRI apparatus as claimed in claim 2, wherein the direction in which light is emitted by the illumination unit makes an included angle with the horizontal direction, the included angle being within the range of 15 degrees to 60 degrees.

5. The MRI apparatus as claimed in claim 2, further comprising reflective film stuck to the cavity wall of the cavity.

6. The MRI apparatus as claimed in claim 1, wherein the illumination unit is disposed on a top surface of the front portion of the bed board, and emits light towards the cavity.

7. The MRI apparatus as claimed in claim 6, wherein the top surface of the front portion of the bed board is provided with a second slot in which the illumination unit is embedded.

8. The MRI apparatus as claimed in claim 1, wherein the illumination unit is disposed at a front bed head in the front portion of the bed board, and emits light towards a cavity wall of the cavity.

9. The MRI apparatus as claimed in claim 1, further comprising:
a second illumination unit disposed on a front and/or rear housing of the frame;
a third illumination unit disposed on the support rail; and/or
a fourth illumination unit disposed on the cavity.

10. The MRI apparatus as claimed in claim 1, wherein the illumination unit is an LED light source.

11. The MRI apparatus as claimed in claim 2 wherein the illumination unit is an LED light source.

12. The MRI apparatus as claimed in claim 6 wherein the illumination unit is an LED light source.

13. A bed board assembly comprising:
a bed board;
a support frame for supporting the bed board and matching a support rail in a cavity, the bed board entering or exiting the cavity moveably via the support rail; and
an illumination unit disposed in a front portion of the bed board.

14. The bed board assembly as claimed in claim 13, wherein the illumination unit is disposed on the left and right sides of the front portion of the bed board, a top surface of the front portion of the bed board, or a front bed head in a front region of the bed board.

15. The bed board assembly as claimed in claim 13 wherein the illumination unit is an LED light source.
